(19) United States
(12) Patent Application Publication
(10) Pub. No.: US 2024/0183817 A1
(43) Pub. Date: Jun. 6, 2024

(54) Solid-state Reference Electrode Based on Polymeric Membrane

(71) Applicant: NOVA BIOMEDICAL CORPORATION, Waltham, MA (US)

(72) Inventors: Michael COOLEY, Sudbury, MA (US); Dieu-Tu NGUYEN, West Roxbury, MA (US); Samantha MOSLEY, Hyde Park, MA (US); Jianhong PEI, Somerville, MA (US); Chung Chang YOUNG, Salem, NH (US)

(21) Appl. No.: 18/284,578

(22) PCT Filed: May 19, 2021
(86) PCT No.: PCT/US2021/033120

§ 371 Date: Sep. 28, 2023

(51) Int. Cl. G01N 27/333; G01N 27/30; G01N 27/327; G01N 33/487

(52) U.S. Cl. CPC G01N 27/3335; G01N 27/301; G01N 27/307; G01N 27/327; G01N 33/48707

(57) ABSTRACT

A solid-state reference electrode for use in whole blood, serum, plasma, and/or other biological fluids that includes an insulating support substrate, an electrically-conductive material disposed on the insulating support substrate where a first portion of the electrically-conductive material is an electrode portion and a second portion of the electrically-conductive material is an electrical contact portion, an electrode-forming insulating layer having an opening forming a well, a metal-metal salt layer disposed in the well over the electrode portion when the electrically-conductive material is not a metal-metal salt, the metal-metal salt layer selected from silver-silver chloride or mercury-mercurous chloride, and a hydrogel polymeric membrane disposed on the metal-metal salt layer forming the solid-state reference electrode, the hydrogel polymeric membrane being a polymeric hydrogel network containing a chloride salt from (i) inorganic salts of chlorides or organic salts of chlorides and (ii) a supporting electrolyte from salts of an anionic species.

### BACKGROUND OF THE INVENTION

### 1. Field of the Invention

The present invention relates generally to a reference electrode. Particularly, the present invention relates to a solid-state reference electrode.

### 2. Description of the Prior Art

For the potentiometric measurement of a broad spectrum of analytes, such systems require a measurement cell that includes an analyte indicating electrode and a reference electrode. For improved potentiometric sensors, the analysis is dependent on the half of the potentiometric sensor that is the reference electrode.

Examples of reference electrodes known in the art include saturated calomel electrodes and silver/silver chloride electrodes. These reference electrodes typically have a liquid junction portion formed in a distal end portion of a tube through which the solution of potassium or sodium chloride is allowed to flow out. When the measurement to be taken is in a living body or body fluid, the use of a saturated calomel electrode is a concern because such an electrode relies on mercury. The outflow of the potassium or sodium chloride solution even in the silver/silver chloride reference electrode has an effect on a living body. The liquid junction portion is formed of a porous material to reduce the amount of outflow, however, fully satisfactory results are not obtained.

Various attempts at re-designing a reference electrode have been proposed. One device discloses a miniature, solid-state, reference electrode in which a small amount of outflow of an internal electrolyte containing a halogen ion is reduced. The device has an electrical conductor consisting of platinum or silver and a sintered body formed on the periphery of the conductor and consisting of silver halide and silver oxide, a water-containing gel surrounding the electrode portion and containing halogen ion, a hollow tubular body accommodating the water-containing gel and having one end closed by a liquid-junction portion that is either a porous ceramic or by a partitioning wall having an ion permeable portion with a predetermined diffusion coefficient and volume.

A more recent reference electrode device is the use of micro- and nanostructured materials. The construction and quality of the reference electrode often influence (i.e. limits) the simplicity, disposability and analytical quality of potentiometric determination. Yet, the classical construction (Ag/AgCl in concentrated KCl) of the reference electrode still dominates in practice.

Major research into the reference electrode has been focused on the elimination of the liquid phase from the sensor making it all-solid-state without deterioration of performance, i.e. potential stability over time.

One such device discloses the incorporation of polypyrrole microvessels into a membrane composition (i.e. PVC) of the reference electrode. The polypyrrole microvessels were prepared by a photopolymerization method. A water-in-chloroform emulsion that contained pyrrole was irradiated to deposit polypyrrole onto aqueous droplets that yielded polypyrrole microcapsules. KCl and AgNO₃ solutions were used to prepare respective emulsions. The polypyrrole microvessels in the reference membrane contained AgCl and solid KCl. Stability data was determined after about 20 days of conditioning of the reference electrodes in 3 M KCl solution. Depending on the electrode layer (i.e. glassy carbon or polyoctylthiphene), the stability in various concentrations of test solutions (i.e. KCl, NaCl, NaNO₃, or KNO₃) were in the range of 4 to 9 mV. It is notable that none of the tests were performed in whole blood and yet the sensors required 20 days of conditioning of the reference electrodes in 3 M KCl solution to perform the stability analysis. It is further notable that the stability was still relatively high, i.e. 4 to 9 mV.

### SUMMARY OF THE INVENTION

When making measurements using a potentiometric sensor, the accuracy of such a measurement is dependent on the stability of the reference electrode, which represents the other half of the potentiometric cell of the sensor. The construction and quality of the reference electrode limits the simplicity, disposability and analytical quality of the potentiometric determination. As described above, the classical construction of the reference electrode (i.e. Ag/AgCl in concentrated KCl) still dominates. In the recent past, there has been much research on eliminating the liquid phase from the sensor and, thus, making it all solid-state without deterioration of performance. Unfortunately, this is relatively difficult to achieve.

The disadvantages of all prior art reference electrodes used for measuring whole blood, serum, plasma, other biological fluids, and/or aqueous solutions are that they either rely on a flowing reference junction to maintain a stable and low junction potential or have relatively large potential differences. Clearly, these prior art reference electrodes are not reliable since long-term storage and fluctuating junction potentials are serious problems that plague these prior art references for use as disposable, single-use, reference electrodes.

It is an object of the present invention to provide a solid-state reference electrode that has relatively stable junction potential.

It is an object of the present invention to provide a solid-state reference electrode that has a relatively minimal residual junction potential.

It is another object of the present invention to provide a solid-state reference electrode that has a relatively stable junction potential in liquids having a wide range of ionic concentrations.

It is a further object of the present invention to provide a solid-state reference electrode that does not need conditioning in a chloride solution before use.

It is yet a further object of the present invention to provide a solid-state reference electrode that has a relatively small volume of reference components.

It is still another object of the present invention to provide a solid-state reference electrode that has a long-term storage capability.

The present invention achieves these and other objectives by providing a solid-state reference electrode having a metal/metal halide electrode covered by a polymeric membrane.

In one embodiment of the present invention, there is disclosed a solid-state reference electrode for use in whole blood, serum, plasma, other biological fluids, and/or aqueous solutions. The solid-state reference electrode includes an insulating support substrate, an electrically-conductive material disposed on the insulating support substrate, an electrode-forming insulating layer with a well, a metal-metal salt layer disposed in the well, a hydrogel polymeric membrane disposed onto the metal-metal salt layer. The electrically-conductive material has a first portion that is an electrode portion and a second portion that is an electrical contact portion. The electrically-conducting material comprises one of a conductive noble metal, an electrically conductive ink, or a metal-metal salt selected from the group consisting of silver-silver chloride, a mixture of silver and silver chloride, a silver metal coated with silver chloride, and mercury-mercurous chloride. The electrode-forming insulating layer has an opening where the insulating layer is disposed onto the insulating substrate layer where the opening forms a well and exposes the electrode portion of the electrically-conductive material. The metal-metal salt layer is disposed in the well over the electrode portion of the electrically-conductive material when the electrically-conductive material is not the metal-metal salt. The hydrogel polymeric membrane is disposed onto the metal-metal salt layer forming the solid-state reference electrode. The well has a predefined volume and exposes the electrode portion of the electrically-conductive material. The hydrogel polymeric membrane forms a polymeric hydrogel network containing (i) a chloride salt and (ii) a supporting electrolyte from salts of an anionic species.

In one embodiment of the present invention, the metal-metal salt is one of silver-silver chloride or mercury-mercurous chloride.

In one embodiment of the present invention, the chloride salt is one of inorganic salts of chlorides or organic salts of chlorides.

In one embodiment of the present invention, the solid-state reference electrode further includes a second polymeric membrane disposed on the hydrogel polymeric membrane where the second polymeric membrane is selected from hydrophilic silicon compounds or from lipophilic polymers.

In one embodiment of the present invention, the conductive noble metal is selected from one of gold, platinum, palladium, copper, indium, and tin oxide.

In one embodiment of the present invention, the chloride salt is selected from the group consisting of sodium chloride, potassium chloride, lithium chloride, choline chloride, 1-butyl-3-methylimidazolium chloride, 1-butyl-2,3-dimethylimidazolium chloride, 1-butyl-1-methylpyrrolidium chloride, 1,2-dimethyl-3-propylimidazolium chloride, and 1,3-dimethylimidazolium chloride.

In one embodiment of the present invention, the supporting electrolyte is selected from the group consisting of lithium, sodium or potassium salts of citrates, acetates, sulfonates or triflates.

In one embodiment of the present invention, the silicone compounds for the second polymeric membrane are selected from the group consisting of diluted silicon tetrachloride, aminopropyltriethoxysilane, n-[3-(trimethoxysilyl)propyl]ethylenediamine, methyltrimethoxy silane and phenyltrimethoxysilane.

In one embodiment of the present invention, the lipophilic polymers are selected from the group consisting of polyvinylpyrrolidone, polyvinyl alcohol, polyvinyl chloride (PVC), poly(methyl methacrylate), agar, gelatin, poly(urethane), cellulose acetate butyrate, cellulose acetate, and nitro cellulose.

In one embodiment of the present invention, the metal-metal salt layer is silver-silver chloride, the chloride salt in the hydrogel polymeric membrane is potassium chloride, and the supporting electrolyte is lithium acetate.

In one embodiment of the present invention, the hydrogel polymeric membrane is selected from the group consisting of polyacrylates, polymethacrylates, polyvinyl compounds, polyurethanes, polycarbamoyl sulfonates, polyureas, polyethers, crosslinkable Polyvinyl alcohol with styrylpyridinium pendent groups (i.e. PVA-SBQ), crosslinked protein matrix like gelatin, silk fibroin, Bis (trimethylsilyl)acetamide (i.e. BSA), crosslinked polysacharrides like cellulose, dextrans, cyclodextrans, alginates, chitosan, agar, and any combination thereof.

In one embodiment, the hydrogel polymeric membrane further includes (a) a hydrophilic plasticizer capable of filing the polymeric hydrogel network and solidifying and plasticizing the hydrogel polymeric membrane, and (b) a high molecular weight polymer capable of reinforcing the polymeric hydrogel network.

In one embodiment, the hydrophilic plasticizer is selected from at least one of glycerol, polyethylene glycol, ethylene glycol monomethyl ester, ethylene glycol, formamide.

In one embodiment, the high molecular weight polymer is one of polyethylene glycol, polyvinyl alcohol, polyvinylpyrrolidone, poly(2-hydroxyethyl methacrylate), and particulates like pyrogenic silica or latex.

In one embodiment, the hydrogel polymeric membrane includes poly-2-hydroxyethyl methacrylate, ethylene glycol as the hydrophilic plasticizer, and polyvinylpyrrolidone as the high molecular weight polymer.

In one embodiment, a method of making a solid-state reference electrode for use in whole blood, serum, plasma, and/or other biological fluids is disclosed. The method includes obtaining either (a) an insulating support substrate having an electrically-conductive material disposed on at least one side of the insulating support substrate, or (b) an insulating support substrate and disposing an electrically-conductive material onto at least one side of the insulating support substrate wherein a first portion of the electrically-conductive material is an electrode portion and a second portion of the electrically-conductive material is an electrical contact portion, the electrically-conducting material in (a) and (b) comprising one of a conductive noble metal, an electrically conductive ink, or a metal-metal salt selected from the group consisting of silver-silver chloride and mercury-mercurous chloride. The method further includes disposing an electrode-forming insulating layer having an opening onto the insulating support substrate where the opening forms a well exposing the electrode portion of the electrically-conductive material, forming a metal-metal salt layer on the portion of the conductive noble metal film exposed in the opening of the electrode-forming insulating layer, disposing a predefined amount of a precursor solution into the well, the precursor solution containing a chloride salt solution, a supporting electrolyte solution, a hydrogel polymer, a hydrophilic plasticizer, a high molecular weight polymer, a cross-linking reagent, and a radical initiator, and exposing the precursor solution in the well to radiation forming a hydrogel polymeric membrane and thereby forming the solid-state reference electrode.

In one embodiment, the forming step includes forming a silver-silver chloride layer.

In one embodiment, the method includes forming the precursor solution by mixing a predefined amount of a chloride salt solution, a predefined amount of a supporting electrolyte solution, a predefined amount of hydroxyethyl methacrylate, a predefined amount of ethylene glycol, a predefined amount of polyvinylpyrrolidone, a predefined amount of tetraehtylene glycol dimethacrylate and a predefined amount of 2,2-dimethoxy-2-phenylacetophenone.

In one embodiment, the method includes forming the precursor solution by mixing selecting an amount in a range consisting of 0.1 mM to saturated, 10 mM to 500 mM and a concentration of 200 mM of the chloride salt, an amount in a range consisting of 10 mM to saturated, 1M to 6 M and 3 M to 5M of the supporting electrolyte salt, an amount in a range consisting of 20-80 wt. %, 30-70 wt. % and 45-55 wt. % of poly-2-hydroxyethyl methacrylate as the cross-linkable hydrogel polymer, an amount in a range consisting of 20-80 wt. %, 30-60 wt. % and 40-50 wt. % of ethylene glycol as the hydrophilic plasticizer, an amount in a range consisting of 0.5-10.0 wt. % and 1-5 wt. % of polyvinylpyrrolidone as the high molecular weight polymer, an amount in a range of 0.1-2.0 wt. % and 0.5-1.0 wt. % of tetraethylene glycol dimethacrylate as the cross-linking reagent, and an amount in the range of 0.01-2.0 wt. % and 0.5-1.0 wt. % of 2,2-dimethoxy-2-phenylacetophenone as the radical initiator.

In one embodiment, there is disclosed a method of making the hydrogel polymeric membrane for use in the solid-state reference electrode. The method includes (a) forming a precursor solution that includes a predefined amount of a chloride salt, a predefined amount of a supporting electrolyte salt, a predefined amount of cross-linkable hydrogel polymer, a predefined amount of hydrophilic plasticizer, a predefined amount of high molecular weight polymer, a predefined amount of a cross-linking reagent, and a predefined amount of a radical initiator, (b) disposing a predefined amount of the precursor solution onto a solid-state electrode containing a metal-metal salt electrolytic electrode, and (c) photo-irradiating the predefined amount of the precursor solution to ultraviolet light.

In one embodiment, the method further includes selecting potassium chloride as the chloride salt, selecting lithium acetate as the supporting electrolyte salt, selecting poly-2-hydroxyethyl methacrylate as the cross-linkable hydrogel polymer, selecting ethylene glycol as the hydrophilic plasticizer, selecting polyvinylpyrrolidone as the high molecular weight polymer, selecting tetraethylene glycol dimethacrylate as the cross-linking reagent, and selecting 2,2-dimethoxy-2-phenylacetophenone as the radical initiator.

In one embodiment, the method further includes selecting an amount of the chloride salt in a range from the group consisting of 0.1 mM to saturated, 10 mM to 500 mM and a concentration of 200 mM of the chloride salt, selecting an amount of the supporting electrolyte salt in a range from the group consisting of 10 mM to saturated, 1M to 6 M and 3 M to 5M, selecting an amount of poly-2-hydroxyethyl methacrylate as the cross-linkable hydrogel polymer in a range from the group consisting of 20-80 wt. %, 30-70 wt. % and 45-55 wt. %, selecting an amount of ethylene glycol as the hydrophilic plasticizer in a range from the group consisting of 20-80 wt. %, 30-60 wt. % and 40-50 wt. %, selecting an amount of polyvinylpyrrolidone as the high molecular weight polymer in a range from the group consisting of 0.5-10.0 wt. % and 1-5 wt. %, selecting an amount of tetraethylene glycol dimethacrylate as the cross-linking reagent in a range from the group consisting of 0.1-2.0 wt. % and 0.5-1.0 wt. %, and selecting an amount of 2,2-dimethoxy-2-phenylacetophenone as the radical initiator in the range from the group consisting of 0.01-2.0 wt. % and 0.5-1.0 wt. %.

### BRIEF DESCRIPTION OF THE DRAWINGS

FIG. 1 is a perspective view of one embodiment of the present invention showing a solid-state reference electrode.

FIG. 1A is a cross-sectional view of the solid-state reference electrode shown in FIG. 1.

FIG. 1B is a cross-sectional view of another solid-state reference electrode showing an optional second polymeric membrane disposed onto the hydrogel polymeric membrane.

FIG. 2 is a perspective view of a potentiometric sensor having the solid-state reference electrode integrated with an ion-selective electrode.

FIG. 2A is a cross-sectional view of the potentiometric sensor of FIG. 2.

FIG. 3 is a perspective view of a potentiometric sensor having the solid-state reference electrode integrated with multiple ion-selective electrodes.

FIG. 3A is a cross-sectional view of the potentiometric sensor of FIG. 3.

FIG. 4 is a cross-sectional view of the solid-state reference electrode showing the electrically-conductive material sandwiched between two electrically-insulating substrates with windows aligned on either side where one window is the electrode portion and the other window is the contact portion.

FIG. 5 is a graphical illustration showing the stability response of the solid-state reference electrode at various concentrations of sodium chloride solution.

FIG. 6 is a graphical illustration showing the stability response of the solid-state reference electrode at various concentrations of calcium chloride solution.

FIG. 7 is a graphical illustration showing the stability response of the solid-state reference electrode across a range of pH value solutions.

FIG. 8 is a graphical illustration showing the response in whole blood for a potentiometric sensor incorporating the solid-state reference electrode and a solid-state potassium ion-selective sensor.

FIG. 9 is a graphical illustration showing the response in whole blood for potentiometric sensor incorporating the solid-state reference electrode and a solid-state calcium ion-selective sensor.

### DETAILED DESCRIPTION OF THE INVENTION

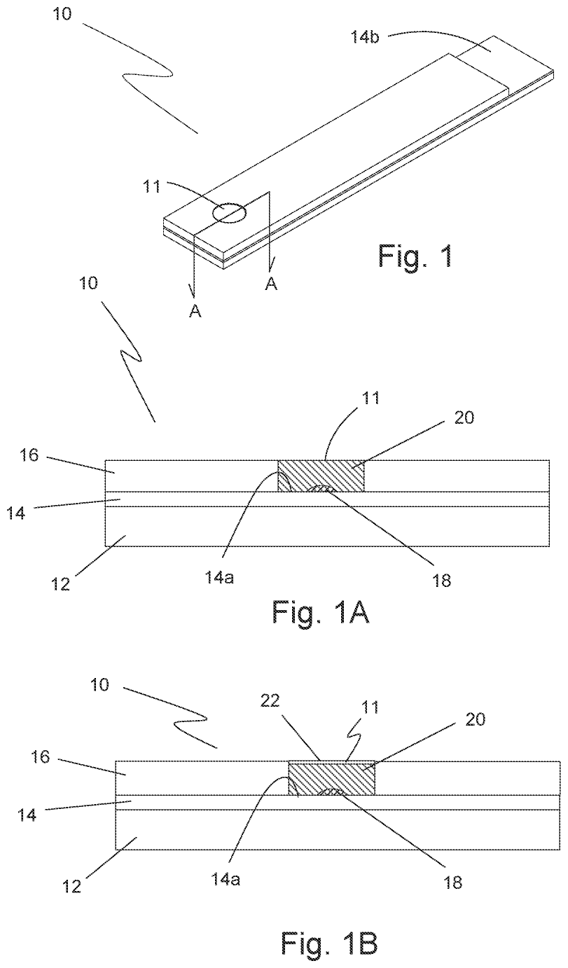

The general construction of solid-state reference electrode will first be described with reference to FIGS. 1, 1A and 1B.

FIG. 1 is a perspective view of one embodiment of a planar, disposable, reference electrode 10 having a solid-state reference electrode 11. FIG. 1A illustrates a cross-sectional view of one embodiment of a solid-state reference electrode 11. Solid-state reference electrode 11 has a planar shape and includes an insulating support substrate 12 (i.e. base insulating layer), an electrically-conductive material 14 disposed on the insulating support 12 where a first portion of the electrically-conductive material is an electrode portion 14a (shown in FIGS. 1A and 1B) and a second portion of the electrically-conductive material is an electrical contact portion 14b (shown in FIG. 1), and an electrode-forming insulating layer 16 having an opening 16a that forms a well 16b having predefined dimensions and exposing a portion of the conductive noble metal film 14. All layers are made of a dielectric material, preferably plastic. Examples of a preferred dielectric material are polyvinyl chloride, polycarbonate, polysulfone, nylon, polyurethane, cellulose nitrate, cellulose propionate, cellulose acetate, cellulose acetate butyrate, polyester, acrylic and polystyrene. The electrically-conductive material may be an electrically-conductive, noble metal film, a metal-metal salt film such as silver-silver chloride, an electrically-conductive ink printed onto the insulating support substrate, or a tracing of the electrically-conductive material. The electrically-conductive material has an electrode portion and a contact portion where the electrical contact portion is used to electrically couple the contact portion to an analyte measuring circuitry.

The thickness of the electrode-forming insulating layer 16 as well as the dimensions of the well 16b define the thickness and aspect ratio of the chemistry that fills the well 16b and thus helps to control the volume and surface area of the electrode and its interfaces, among other details. The preferred thickness of the electrode-forming insulating layer 16 is 0.004 inches (0.1 mm) and the preferred diameter of opening 16a is 0.035 inches (0.89 mm). This creates a well 16b with a fill volume of about 0.033 uL (33 nL). Electrode-forming insulating layer 16 is preferably a medical grade, one-sided adhesive tape available from Adhesive Research, Inc., of Glen Rock, PA or Global Instrument Corporation (GIC) (Taiwan). Acceptable thicknesses of the tape for use in the present invention are in the range of about 0.001 in. (0.025 mm) to about 0.005 in. (0.13 mm). The preferred thickness is about 0.004 in. (0.1 mm). It should be understood that the use of a tape is not required. Electrode-forming insulating layer 16 may be made from a plastic sheet and may be coated with a pressure sensitive adhesive, a photopolymer, ultrasonically-bonded to insulating support substrate 12, silk-screened onto insulating support substrate 12, or 3D printed onto insulating support substrate 12 to achieve the same results as using the polyester tape mentioned.

A metal-metal salt layer (M/MX) 18 is disposed on the electrode portion of the conductive noble metal film 14 forming an electrolytic electrode and may cover all of the exposed portion of the conductive noble metal film 14 or only a portion of the exposed portion of the conductive noble metal film 14. A hydrogel polymeric membrane 20 is disposed on the metal-metal salt layer 18 and fills well 16b. It should be remembered that the metal-metal salt layer is not used when the electrically-conductive material is the metal-metal salt but is used when the electrically-conductive material is not the meta-metal salt.

Usable materials for the noble metal film 14 include, but are not limited to, gold, platinum, palladium, copper, indium, tin oxide, etc. In the present invention, the preferred noble metal film 14 is a gold film. Typically, the noble metal film 14 is evaporated on the insulating support substrate 12 or spot coated on the insulating support substrate 12. When a potentiometric sensor 30 as described later is manufactured, the noble metal film 14 is delineated into different electrically-conductive paths, which are electrically isolated from each other. Such electrically-conductive paths may be formed by scribing or scoring the noble metal film 14.

The electrolytic electrode 19 typically develops a referenced potential through insoluble silver or mercury salts that are in contact with their salts. In the present invention, a silver/silver chloride (Ag/AgCl) electrolytic electrode is preferred. There are different methods of making a Ag/AgCl electrolytic electrode. One method is to use a Ag/AgCl ink, but other methods are also acceptable. When the ink-based method is used, a drop of commercially available silver/silver chloride (Ag/AgCl) ink is dispensed and thermally cured onto the gold film 14 to serve as the metal-metal salt 18 based electrolytic electrode. Other methods to create a Ag/AgCl transducer is to dispense a drop of Ag ink or silver epoxy onto the gold surface and to thermally cure the ink or silver epoxy to form silver on the surface. Then the Ag surface is chloridized to form AgCl. In order to maintain a constant potential of the electrolytic electrode, a constant activity of Cl⁻ ion is typically used.

FIG. 1B illustrates a cross-sectional view of another embodiment of a solid-state reference electrode 11. This embodiment like the one illustrated in FIG. 1A has all of the same components except for an optional second polymeric membrane 22 disposed on the hydrogel polymeric membrane 20 creating a double-layered solid-state reference electrode. This optional second polymeric membrane 22 extends the lifetime and stability of the electrode in sample solutions. Additional advantages of the optional second polymeric membrane 22 includes enhancing adhesion between the polymeric reference electrode membrane 20 and the electrolytic electrode 19, and also eliminates electrode fouling and increases the electrode long-term stability. A suitable material for the optional second polymeric membrane 22 is selected from, but not limit to, a group of the silicon compounds such as diluted silicon tetrachloride, aminopropyltriethoxysilane, n-[3-(trimethoxysilyl)propyl]ethylenediamine, methyltrimethoxy silane and phenyltrimethoxysilane; or from other lipophilic polymers such as polyvinylpyrrolidone, polyvinyl alcohol, PVC, poly(methyl methacrylate), agar, gelatin, poly(urethane), cellulose acetate butyrate, cellulose acetate, nitro cellulose, etc. In this application, cellulose acetate butyrate was used and the concentration is preferably in the range of 0.001 to 5%, more preferable at 0.02-1%, most preferable at 0.05 to 0.2%.

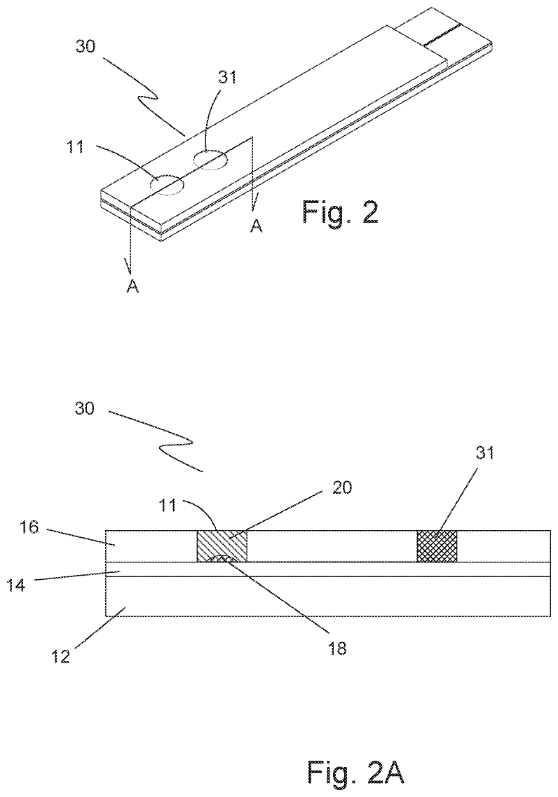

Turning now to FIGS. 2 and 2A, there is illustrated an example of a potentiometric sensor 30 incorporating the monolayered solid-state reference electrode 11 and an ion-selective electrode 31. The ion-selective electrode 31 may be one capable of measuring ions in a sample such as Na⁺, K⁺, Ca²⁺, Mg²⁺, F⁻, Cl⁻, and the like. FIG. 2 illustrates one example of the potentiometric sensor 30 while FIG. 2A is a cross-sectional view of the monolayered solid-state reference electrode 11 as described above and shown in FIG. 1A, and an ion-selective electrode 31.

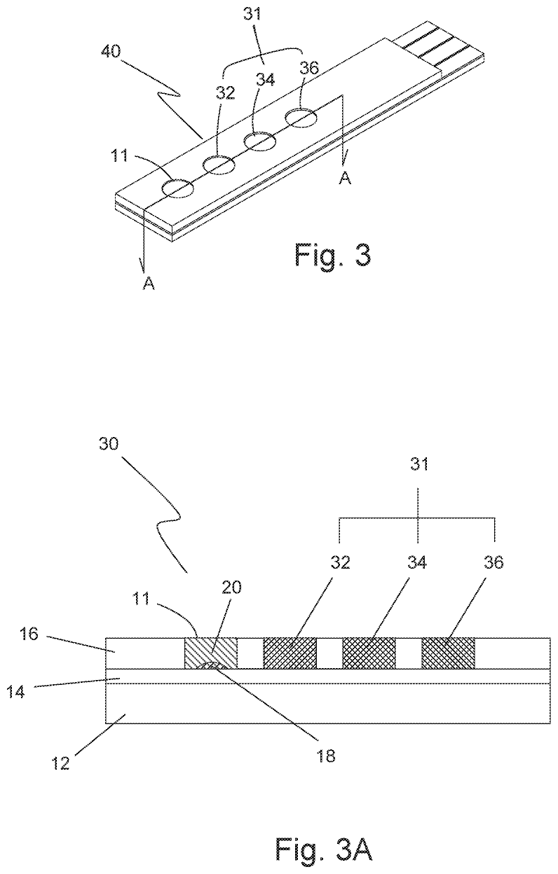

FIGS. 3 and 3A illustrate one example of a multi-potentiometric sensor 40 incorporating the monolayered solid-state reference electrode 11 and multiple ion-selective electrodes 31. As described for FIGS. 2 and 2A, each of the ion-selective electrodes 31 is capable of measuring a particular ion in the sample such as Na⁺, K⁺, Ca²⁺, Mg²⁺, F⁻, Cl⁻, and the like. FIG. 3 illustrates one example of the multi-potentiometric sensor 40 while FIG. 3A is a cross-sectional view of the monolayered solid-state reference electrode 11 and three ion-selective electrodes 32, 34 and 36 where each of the three ion-selective electrodes 32, 34, and 36 may measure a different ionic species such as, for example, a Na⁺ ion, K⁺ ion, Ca²⁺ ion, etc.

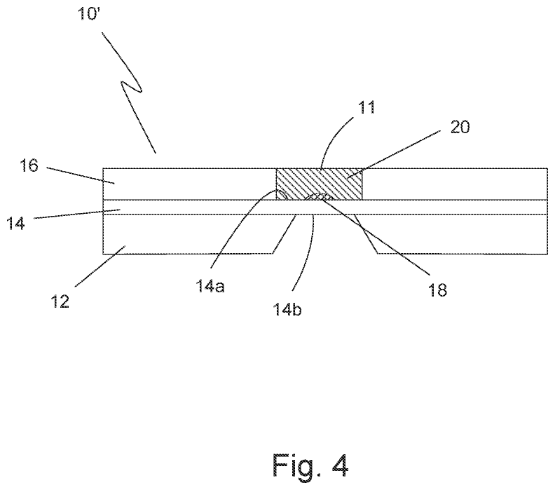

FIG. 4 illustrates another embodiment of a planar, disposable, reference electrode 10′. In this embodiment, the structure of the disposable reference 10′ is similar to the disposable reference electrode 10 but with a structural difference. The difference lies in the location of the electrical contact portion. In this embodiment, the electrical contact portion of the electrically-conductive material is on the opposite side of the electrode portion and not co-planar with the electrode portion. The insulating support substrate also has an opening that exposes the electrical contact portion to allow electrically coupling the disposable, solid-state reference electrode to an analyte measuring circuitry.

### Polymeric Membrane Precursor

A UV curable hydrogel polymer-based membrane precursor solution mixed with salt of a chloride solution (KCl here) where the chloride as the ion source will participate with the Ag/AgCl electrode and the second supporting electrolyte (lithium acetate), which is used to eliminate the liquid junction potential and to maintain the activity of chloride ion. This mixed precursor solution is dropped on the top of the Ag/AgCl electrode surface. The solution is then photo polymerized into polymeric hydrogel by exposing it to 365 um UV light at 18 mW/cm² for 5 minutes under nitrogen environment with no more than 2% oxygen in the environment.

### Ingredients of Precursor Solution

The ingredients of the precursor solution include a chloride salt solution, a supporting electrolyte compound solution, a monomer, a hydrophilic plasticizer, a hydrophilic polymer, a cross-linking reagent, and a radical initiator.

### Chloride Salt Solution

In the present invention, the salt of chloride compounds includes, but is not limited to, inorganic salts of chlorides such as sodium chloride, potassium chloride, lithium chloride, et. al. or organic salts of chlorides, such as choline chloride, 1-butyl-3-methylimidazolium chloride, 1-butyl-2,3-dimethylimidazolium chloride, 1-butyl-1-methylpyrrolidium chloride, 1,2-dimethyl-3-propylimidazolium chloride, 1,3-dimethylimidazolium chloride, et. al. The salt used in the solid-state reference electrode of the present invention for illustration purposes is KCl. The concentration of KCl is in the range of 0.1 mM to saturated solution, preferable in the range of 10 mM to 500 mM, and more preferable with a concentration of 200 mM.

### Supporting Electrolyte

Because the volume of the hydrogel polymeric membrane 20 is so small and in order to maintain a good performance of the solid-state reference electrode and eliminate the liquid junction potential, another supporting electrolyte is used in the present invention. The supporting electrolyte compound solution is added to maintain a constant liquid junction potential. The supporting electrolyte is chosen from lithium, sodium or potassium salts of anionic species, such as citrates, acetates, sulfonates, triflates or similar compounds. In the present invention, lithium acetate is used in the present invention for illustration purposes. The concentration of lithium acetate used is in a range of 10 mM to saturated solution, preferably in a range of 1 M to 6.0 M, and more preferably in a range of 3 M to 5 M. Where the Nernst equation deals with “activity” of chloride ions rather than concentration, it is possible to achieve potentials comparable to conventional saturated KCl electrodes, provided that the hydrogel polymeric membrane 20 itself contains sufficient salt. Lithium acetate was found to be more soluble in the polymeric membrane precursor mixture used in forming the hydrogel polymeric membrane 20.

### Monomer

The monomer is a hydrogel polymer that forms a hydrogel porous polymeric matrix, which is a cross-linked polymer network, that can swell with water. The swelling properties of hydrogels can be tailored by controlling the porosity and hydrophilic properties of the polymer. While any polymer hydrogel matrix can be used, including, but not limited to, polyacrylates, polymethacrylates, polyvinyl compounds, polyurethanes, polycarbamoyl sulfonates, polyureas, polyethers, cross-linkable PVA-SBQ, or even a crosslinked protein matrix like gelatin, silk fibroin, BSA, etc., or crosslinked polysacharrides like cellulose, dextrans, cyclodextrans, alginates, chitosan, Agar, etc., or any combination of these as composite materials, poly-2-hydroxyethyl methacrylate (pHEMA) was selected to demonstrate the present invention. Poly-2-hydroxyethyl methacrylate is made from the monomer hydroxyethyl methacrylate (HEMA). The concentration of HEMA is in the range of 20% to 80%, preferably in the range of 30% to 70% and more preferably in the range of 45% to 55%. Poly-2-hydroxyethyl methacrylate was selected based on its unique properties of being a stable methacrylate backbone with neutral, non-ionic, hydrophilic hydroxyethyl ligands, is non-toxic and biocompatible with anti-fouling properties, making it usable in testing blood and serum samples. Because it is made from HEMA, the HEMA monomer solution is easily dispensed into the well 16b, taking the shape of well 16b, and forming to the topography of the substrate/Au/AgCl surfaces creating good adhesion to the various surfaces of the substrate and creating stable interfaces.

### Hydrophilic Plasticizer

In the dry state of pHEMA, the so called “xerogel” state, the pHEMA gel is not flexible. Because of the more hydrophobic methacrylate backbone, it takes a relatively long time for the dry pHEMA xerogel to absorb enough water to fill its capacity. This long absorption time is not a very good characteristic for something that needs to wet-up relatively quickly and function right out of the package. The hydrophilic plasticizer fills the polymeric hydrogel network, and effectively solidifies and plasticizes the gel. The hydrophilic plasticizer also allows ions to pass through the membrane. The hydrophilic plasticizer is selected from the group including glycerol, polyethylene glycol, ethylene glycol monomethyl ester, ethylene glycol, formamide, and the like. These substances are low molecular weight, hygroscopic materials that can fill the pores of the hydrogel polymeric network, effectively plasticizing the gel. Further, they do not evaporate away and remain in the gel structure during storage. Ethylene glycol (EG) was chosen in the present invention for demonstration purposes because ethylene glycol is closest to water in size and density. The hydrophilic plasticizer puts the hydrogel in a swollen-flexible state, allowing the polymer to react with samples more readily as they enter the gel structure. As anti-freeze agents, these hydrophilic plasticizers further help protect the hydrogel from changes in low temperature storage. The amount of hydrophilic plasticizer in the formulation will depend on the configuration of the sensor and design of the substrate.

A further role of hydrophilic plasticizer in the formula is as a hydrogen bond donor that can dissociate lithium acetate, which acts as a hydrogen bond acceptor in this case. This combination is an ionically conductive liquid at room temperature that fills the pores of the hydrogel matrix and helps to stabilize the membrane potential at the sample-to-gel interface and ensures that the double layer close to the electrode surface has a high metal ion concentration. An additional benefit of using ethylene glycol (EG) is that the equally-transferring salt (LiAc in this invention) is soluble in EG and forms the ionically conductive liquid that fills the pores of the hydrogel matrix. The concentration of ethylene glycol is in the range of 20% to 80%, preferably in the range of 30% to 60%, and more preferably in the range of 40% to 50%.

### Hydrophilic Polymer

Using low molecular weight, hydrophilic plasticizers like ethylene glycol or propylene glycol, which have very low viscosity, low surface tension, and would wet to the substrate too easily, creates thin layers that are not very reproducible when dispensed. An inert, high molecular weight “filler” is needed to increase viscosity and keep the solution in place once applied to the substrate and to enhance the adhesion to the electrode surface. Hydrophilic neutral polymers such as PEG, PVA, PVP, and even pHEMA itself as well as particulates like fumed silica or latex can all be used for this purpose. For demonstration purposes of the present invention, PVP was selected since it dissolved more readily into HEMA monomer than other materials. The long polymer chain entangles into the pHEMA matrix so it won't leech out over time. It also helps to re-enforce the structure of the gel. The concentration of PVP (specifically PVP K90) is in the range of 0.05% to 10%, and preferably in the range of 1% to 5%.

### Cross-Linking Agent

Crosslinking reagents contain at least two reactive groups that will connect themselves to the functional groups such as primary amines, sulfhydryls, carbonyls, carbohydrates and carboxylic acids. In this application, tetraethylene glycol dimethacrylate (TEGDMA) was used as the crosslinking reagent. TEGDMA is a bifunctional methacrylate molecule which can be used as a cross-linking agent in free radical chain polymerization to form a 3D gel structure. TEGDMA has a low volatility and is a non-flammable product with high solubility in water. The concentration of the cross-linking reagent is in a range of 0.1% to 2%, and more preferably in a range of 0.5% to 1%.

### Radical Initiator

Photoinitiated polymerization is one of the polymerization techniques of monomers, where the polymerization is initiated by reactive species such as radicals, cations, or anions that can be generated by photo-irradiation. Liquid monomers can be turned to a solid or semi-solid by the polymerization. A photoinitiator is a molecule that creates reactive species (free radicals, cations or anions) when exposed to radiation (UV or visible). Synthetic photoinitiators are key components in photopolymers. 2,2-Dimethoxy-2-phenylacetophenone (DMPA) is a photoinitiator, which is used to initialize radical chain polymerization e.g. in the preparation of acrylate polymers. Under the influence of UV light, the molecule will form radicals which initiate the radical polymerization, and was used in the present invention. The concentration of the radical initiator is in a range of 0.01% to 2%, and more preferably in a range of 0.5% to 1%.

Table 1 illustrates an example of a precursor solution for making the hydrogel polymeric membrane 20.

TABLE 1
Reference Membrane for SSRE
pHEMA Precursor Solution

| Chemical | Weight(g) | Wt % |
|---|---|---|
| HEMA | 5.755 | 51.36 |
| EG | 5.000 | 44.62 |
| TEGDMA | 0.100 | 0.89 |
| DMPA | 0.100 | 0.89 |
| PVP K90 | 0.250 | 2.23 |
| Total | 11.205 | |

| Chemical | Concentration |
|---|---|
| Li-Acetate | 4.5M |
| KCl | 200 mM |

Turning now to FIG. 4, there is illustrated a graphical illustration showing the stability response at various concentrations of a NaCl solution as measured between the solid-state reference electrode versus a commercially available reference electrode (saturated calomel electrode (SCE)). The solid-state reference electrode of present invention is used as the working electrode. The results show that the potential of the solid-state hydrogel membrane reference electrode of the present invention is not affected by the concentration of NaCl in the sample solution and shows good stability over a very large concentration range.

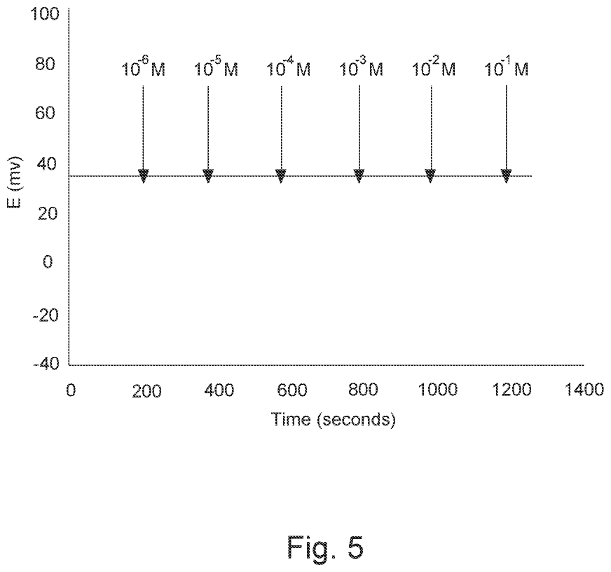

FIG. 5 is a graphical illustration showing the stability response at various concentrations of a CaCl₂) solution as measured between the solid-state reference electrode versus a commercially available reference electrode (saturated calomel electrode (SCE)). The solid-state reference electrode of present invention is used as the working electrode. The results show that the potential of the solid-state hydrogel membrane reference electrode of the present invention is not affected by the concentration of CaCl₂) in the solution and shows good stability over a very large concentration range.

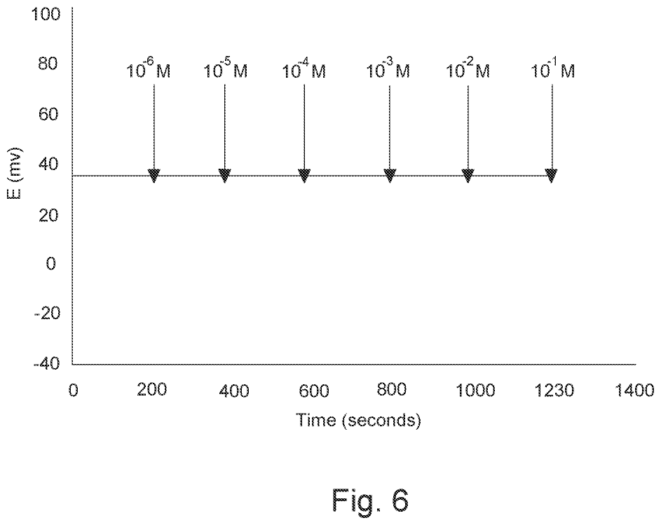

FIG. 6 is a graphical illustration showing the stability response across a range of pH value solutions as measured between the solid-state reference electrode versus a commercially available reference electrode (saturated calomel electrode (SCE)). The solid-state reference electrode of present invention is used as working electrode. The results show that the potential of the solid-state hydrogel membrane reference electrode of the present invention is not affected by the pH values of the solution and shows good stability over a very large pH range.

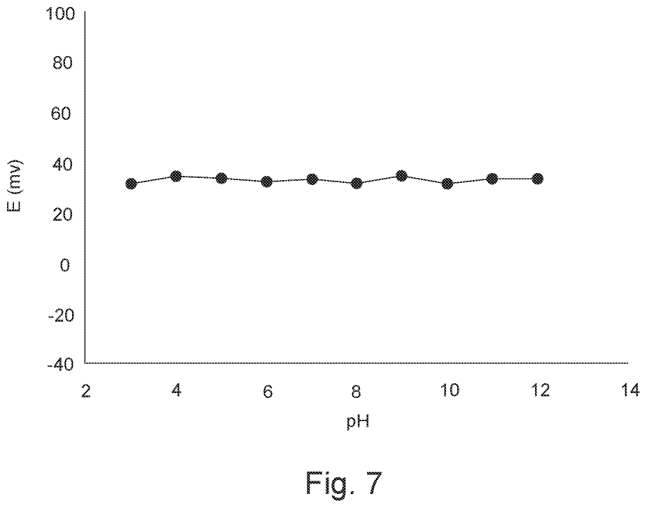

FIG. 7 is a graphical illustration showing the response to potassium ion concentration as measured between the solid-state reference electrode of the present invention and a potassium ion selective electrode. The solid-state reference electrode of present invention is used as the reference electrode and a potassium ion selective electrode is used as the working electrode. The output of the potential response E (mV) of the potassium ion selective electrode versus the log of potassium concentration shows good linearity response with a slope 55.8 mv.

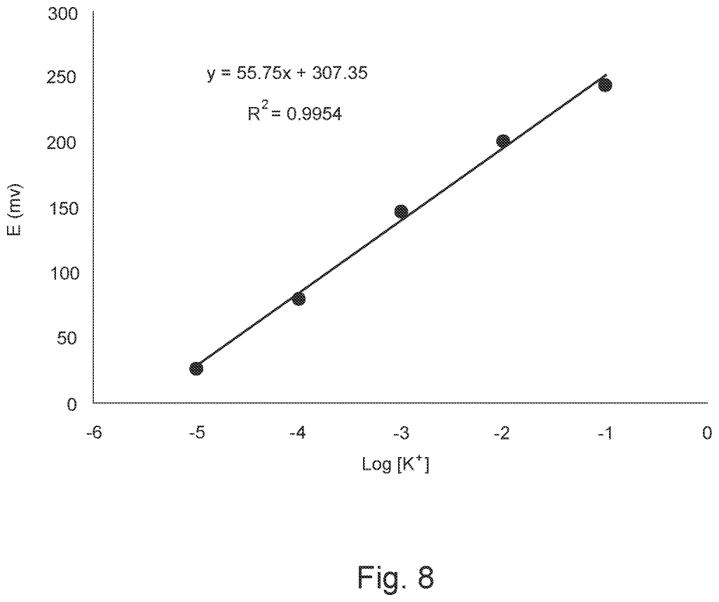

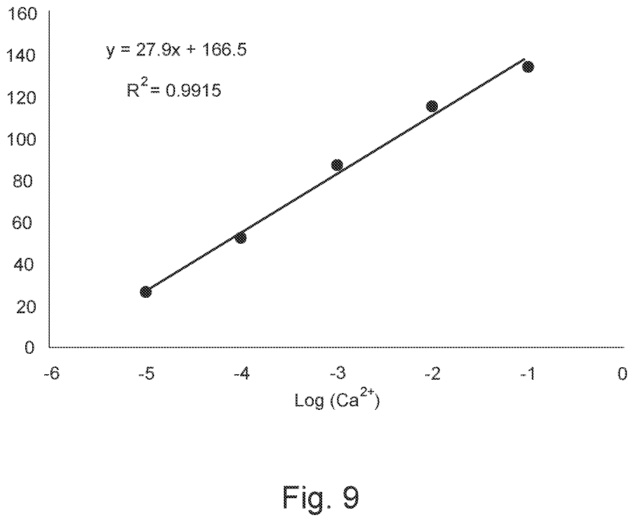

FIG. 8 is a graphical illustration showing the response to calcium ion concentration as measured between the solid-state reference electrode of the present invention and a calcium ion selective electrode. The solid-state reference electrode of present invention is used as the reference electrode and a calcium ion selective electrode is used as the working electrode. The output of the potential response E (mV) of the calcium ion selective electrode versus the log of calcium concentration shows good linearity response with a slope of 27.9 mv.

### Precision in Whole Blood Samples

In all measurements made using the solid-state reference electrode of the present invention, no conditioning (i.e. hydrating) of the solid-state reference electrode was performed or required before any of the tests were performed. The reference in the Nova Biomedical analyzer (Nova Stat Profile Prime) is a flowing reference. The following data tables show the reliability and accuracy of the present invention in whole blood, serum, plasma and/or other biological fluids using the solid-state reference electrode directly from dry storage without pre-conditioning (i.e. hydrating) of the solid-state reference compared to the Nova Biomedical analyzer reference.

Table 2 shows the response for potassium ion reproducibility measurements by a potentiometric sensor incorporating a potassium ion-selective working electrode and a reference electrode of the present invention that is a solid-state hydrogel membrane reference. Twenty (20) potassium sensors 30 similar to that illustrated in FIGS. 2, 2A were used to measure a whole blood sample having a concentration of 4.58 mmol/L of potassium ion. As shown, the mean of the 20 measurements was 4.63 mmol/L of potassium with a coefficient of variation of 3.6%

TABLE 2

| | | | | | Mean | | |
|---|---|---|---|---|---|---|---|
| potassium measurements | | | | | (mM) | CV % | Reference |
| 4.75 | 4.54 | 4.82 | 4.75 | 4.47 | 4.63 | 3.60 | 4.58 |
| 4.47 | 4.88 | 4.64 | 4.62 | 4.77 | | | |
| 4.45 | 4.94 | 4.51 | 4.59 | 4.53 | | | |
| 4.79 | 4.32 | 4.42 | 4.63 | 4.64 | | | |

Table 3 shows the response or sodium ion reproducibility measurements by a potentiometric sensor incorporating a sodium ion-selective working electrode and a reference electrode of the present invention that is a solid-state hydrogel membrane reference. Twenty (20) sodium sensors 30 similar to that illustrated in FIGS. 2, 2A were used to measure a whole blood sample having a concentration of 143.5 mmol/L of sodium ion. As shown the mean of the 20 measurements was 144.8 mmol/L of sodium with a coefficient of variation of 1.7%.

TABLE 3

| | | | | | Mean | | |
|---|---|---|---|---|---|---|---|
| Sodium measurements | | | | | (mM) | CV % | Reference |
| 143.5 | 143.4 | 146.5 | 144.2 | 143.5 | 144.8 | 1.72 | 143.5 |
| 147.1 | 149.3 | 141.2 | 144.7 | 145.1 | | | |
| 149.7 | 143.5 | 146.7 | 142.6 | 142.8 | | | |
| 148.8 | 146.2 | 142.8 | 141.9 | 143.1 | | | |

Table 4 lists potassium measurement tests on 30 different whole blood samples. The reference values for each of the 30 different whole blood samples were measured using a Nova Biomedical Analyzer, Model Stat Profile Prime. It is important to note that no membrane conditioning was performed before measurement of the whole blood samples. The measurement difference is the difference in mmol/L between the value measured by the Nova Biomedical Analyzer and the value measured by the potassium ion-selective sensor incorporating the solid-state reference electrode of the present invention. The Percent Bias is determined by taking the absolute value of the difference between the millimole value of Nova result and the millimole value using the solid-state Reference result and dividing by the millimole value of the Nova result. For single charge ions such as sodium and potassium, one millivolt is equal to a four percent (4%) bias. As shown in the table, the measurement results taken with a potassium ion-selective sensor incorporating the solid-state reference of the present invention indicates that the average value of the Percent Bias for the 30 samples is three percent (3%) and the average millivolt value of that Percent Bias is 0.703 mv. It is interesting to note that for the 30 samples, the maximum value of the Percent Bias is 7% and the maximum value of the millivolt difference is 1.813 mv. This illustrates the accuracy and reliability of the disposable strip sensor incorporating the solid-state reference electrode of the present invention when measuring potassium concentration in a whole blood sample.

TABLE 4
Comparison Results for Potassium Whole Blood Measurements

| | Nova | Reference | Percent Bias | |
|---|---|---|---|---|
| Sample | results | results | (absolute | Millivolt |
| # | (mM) | (mM) | value) | Difference |
| 1 | 3.59 | 3.36 | 6% | 1.602 |
| 2 | 5.32 | 5.36 | 1% | 0.188 |
| 3 | 4.66 | 4.88 | 5% | 1.180 |
| 4 | 4.51 | 4.67 | 4% | 0.887 |
| 5 | 4.45 | 4.38 | 2% | 0.393 |
| 6 | 4.61 | 4.43 | 4% | 0.976 |
| 7 | 3.96 | 3.87 | 2% | 0.568 |
| 8 | 5.54 | 5.61 | 1% | 0.316 |
| 9 | 4.11 | 4.22 | 3% | 0.669 |
| 10 | 5.61 | 5.47 | 2% | 0.624 |
| 11 | 5.33 | 5.23 | 2% | 0.469 |
| 12 | 4.91 | 5.04 | 3% | 0.662 |
| 13 | 4.31 | 4.22 | 2% | 0.522 |
| 14 | 5.02 | 4.89 | 3% | 0.647 |
| 15 | 4.71 | 4.82 | 2% | 0.584 |
| 16 | 5.03 | 5.11 | 2% | 0.398 |
| 17 | 5.61 | 5.52 | 2% | 0.401 |
| 18 | 4.55 | 4.44 | 2% | 0.604 |
| 19 | 3.9 | 3.79 | 3% | 0.705 |
| 20 | 5.41 | 5.26 | 3% | 0.693 |
| 21 | 5.08 | 5.19 | 2% | 0.541 |
| 22 | 5.56 | 5.38 | 3% | 0.809 |
| 23 | 4.52 | 4.29 | 5% | 1.272 |
| 24 | 4.38 | 4.33 | 1% | 0.285 |
| 25 | 3.77 | 3.58 | 5% | 1.260 |
| 26 | 5.42 | 5.62 | 4% | 0.923 |
| 27 | 5.27 | 5.12 | 3% | 0.712 |
| 28 | 3.31 | 3.55 | 7% | 1.813 |
| 29 | 4.64 | 4.52 | 3% | 0.647 |
| 30 | 5.32 | 5.23 | 2% | 0.423 |

| | | |
|---|---|---|
| Average Value | 3% | 0.703 |
| Maximum Value | 7% | 1.813 |

Table 5 lists sodium measurement tests on 30 different whole blood samples. The reference values for each of the 30 different whole blood samples were measured using a Nova Biomedical Analyzer, Model Stat Profile Prime. The measurement difference is the difference in mmol/L between the value measured by the Nova Biomedical Analyzer and the value measured by the sodium ion-selective sensor incorporating the solid-state reference electrode of the present invention. As previously stated above, the Percent Bias is determined by taking the absolute value of the difference between the millimole value of Nova result and the millimole value using the solid-state Reference result and dividing by the millimole value of the Nova result. For single charge ions such as sodium and potassium, one millivolt is equal to a four percent (4%) bias. As shown in the table, the measurement results taken with a sodium ion-selective sensor 30 shown in FIGS. 2-2A incorporating the solid-state reference of the present invention indicates that average value of the Percent Bias for the 30 samples is one percent (1%) and the average millivolt value of that Percent Bias is 0.008 mv. It is interesting to note that for the 30 samples, the maximum value of the Percent Bias is 2% and the maximum value of the millivolt difference is 0.481 mv. This illustrates the accuracy and reliability of the disposable strip sensor incorporating the solid-state reference electrode of the present invention when measuring sodium concentration in a whole blood sample.

TABLE 5
Comparison Results for Sodium Whole Blood Measurements

| | Nova | Reference | Percent Bias | |
|---|---|---|---|---|
| Sample | results | results | (absolute | Millivolt |
| # | (mM) | (mM) | value) | Difference |
| 1 | 153.6 | 152.7 | 1% | 0.146 |
| 2 | 137.4 | 138.6 | 1% | 0.218 |
| 3 | 150.5 | 151.8 | 1% | 0.216 |
| 4 | 147.3 | 145.8 | 1% | 0.255 |
| 5 | 126.8 | 128.4 | 1% | 0.315 |
| 6 | 144.5 | 142.8 | 1% | 0.294 |
| 7 | 155.9 | 152.9 | 2% | 0.481 |
| 8 | 147.6 | 147.2 | 0% | 0.068 |
| 9 | 156.8 | 157.3 | 0% | 0.080 |
| 10 | 149.1 | 148.9 | 0% | 0.034 |
| 11 | 141.7 | 143.1 | 1% | 0.247 |
| 12 | 144.1 | 142.7 | 1% | 0.243 |
| 13 | 136 | 137.3 | 1% | 0.239 |
| 14 | 137.3 | 136.9 | 0% | 0.073 |
| 15 | 131.5 | 130.5 | 1% | 0.190 |
| 16 | 158.8 | 159.1 | 0% | 0.047 |
| 17 | 148.9 | 147.5 | 1% | 0.235 |
| 18 | 140.6 | 140.9 | 0% | 0.053 |
| 19 | 136.3 | 135.2 | 1% | 0.202 |
| 20 | 143.8 | 141.7 | 1% | 0.365 |
| 21 | 135.1 | 137.2 | 2% | 0.389 |
| 22 | 140.4 | 138.7 | 1% | 0.303 |
| 23 | 153 | 151.7 | 1% | 0.212 |
| 24 | 136.8 | 138.1 | 1% | 0.238 |
| 25 | 137.9 | 136.2 | 1% | 0.308 |
| 26 | 146.2 | 145.4 | 1% | 0.137 |
| 27 | 150.9 | 151.4 | 0% | 0.083 |
| 28 | 133 | 132.6 | 0% | 0.075 |
| 29 | 143.6 | 141.8 | 1% | 0.313 |
| 30 | 135.1 | 134.2 | 1% | 0.167 |

| | | |
|---|---|---|
| Average Value | 1% | 0.008 |
| Maximum Value | 2% | 0.481 |

Table 6 lists calcium measurement tests on 30 different whole blood samples. The reference values for each of the 30 different whole blood samples were measured using a Nova Biomedical Analyzer, Model Stat Profile Prime. The measurement difference is the difference in mmol/L between the value measured by the Nova Biomedical Analyzer and the value measured by the calcium ion-selective sensor incorporating the solid-state reference electrode of the present invention. As previously stated above, the Percent Bias is determined by taking the absolute value of the difference between the millimole value of Nova result and the millimole value using the solid-state Reference result and dividing by the millimole value of the Nova result. For double charge ions such as calcium, one millivolt is equal to an eight percent (8%) bias. As shown in the table, the measurement results taken with a calcium ion-selective sensor 30 shown in FIGS. 2-2A incorporating the solid-state reference of the present invention indicates that average value of the Percent Bias for the 30 samples is five percent (5%) and the average millivolt value of that Percent Bias is 0.668 mv. It is interesting to note that for the 30 samples, the maximum value of the Percent Bias is 11% and the maximum value of the millivolt difference is 1.389 mv. This illustrates the accuracy and reliability of the disposable strip sensor incorporating the solid-state reference electrode of the present invention when measuring calcium concentration in a whole blood sample.

TABLE 6
Comparison Results for Calcium Whole Blood Measurements

| | Nova | Reference | Percent Bias | |
|---|---|---|---|---|
| Sample | results | results | (absolute | Millivolt |
| # | (mM) | (mM) | value) | Difference |
| 1 | 1.15 | 1.09 | 5% | 0.652 |
| 2 | 1.45 | 1.51 | 4% | 0.517 |
| 3 | 1.47 | 1.42 | 3% | 0.425 |
| 4 | 1.18 | 1.21 | 3% | 0.318 |
| 5 | 1.2 | 1.15 | 4% | 0.521 |
| 6 | 1.08 | 1.01 | 6% | 0.810 |
| 7 | 1.28 | 1.16 | 9% | 1.172 |
| 8 | 1.26 | 1.12 | 11% | 1.389 |
| 9 | 1.24 | 1.32 | 6% | 0.806 |
| 10 | 1.2 | 1.28 | 7% | 0.833 |
| 11 | 1.26 | 1.31 | 4% | 0.496 |
| 12 | 1.17 | 1.22 | 4% | 0.534 |
| 13 | 1.19 | 1.11 | 7% | 0.840 |
| 14 | 1.18 | 1.1 | 7% | 0.847 |
| 15 | 1.31 | 1.23 | 6% | 0.763 |
| 16 | 1.19 | 1.14 | 4% | 0.525 |
| 17 | 1.27 | 1.21 | 5% | 0.591 |
| 18 | 1.44 | 1.47 | 2% | 0.260 |
| 19 | 1.21 | 1.11 | 8% | 1.033 |
| 20 | 1.24 | 1.31 | 6% | 0.706 |
| 21 | 1.01 | 1.04 | 3% | 0.371 |
| 22 | 1.36 | 1.29 | 5% | 0.643 |
| 23 | 1.32 | 1.41 | 7% | 0.852 |
| 24 | 1.12 | 1.18 | 5% | 0.670 |
| 25 | 1.25 | 1.22 | 2% | 0.300 |
| 26 | 1.35 | 1.41 | 4% | 0.556 |
| 27 | 1.49 | 1.52 | 2% | 0.252 |
| 28 | 1.25 | 1.32 | 6% | 0.700 |
| 29 | 1.36 | 1.25 | 8% | 1.011 |
| 30 | 1.18 | 1.12 | 5% | 0.636 |

| | | |
|---|---|---|
| Average Value | 5% | 0.668 |
| Maximum Value | 11% | 1.389 |

Tables 7-11 lists sodium measurement tests in ten (10) whole blood samples showing the effect of various storage scenarios on sodium sensors incorporating the solid-state reference of the present invention. The reference values for each of the 10 different whole blood samples were measured using a Nova Biomedical Analyzer, Model Stat Profile Prime. The solid-state reference was used directly from storage without any conditioning (i.e. hydrating) of the solid-state reference before use. The measurement difference is the difference in mmol/L between the value measured by the Nova Biomedical Analyzer and the value measured by the sodium ion-selective sensor incorporating the solid-state reference electrode of the present invention. As previously stated above, the Percent Bias is determined by taking the absolute value of the difference between the millimole value of Nova result and the millimole value using the solid-state Reference result and dividing by the millimole value of the Nova result. For single charge ions such as sodium and potassium, one millivolt is equal to a four percent (4%) bias. The results include data of newly-made sensors (Table 7), sensors stored for 6 months at room temperature (Table 8), sensors stored for 6 months at 4° C. (Table 9), sensors stored for 1 year at room temperature (Table 10), and sensors stored for 1 year at 4° C. (Table 11). As shown in the tables, the measurement results taken with a sodium ion-selective sensor 30 shown in FIGS. 2-2A incorporating the solid-state reference of the present invention indicates that the average millivolt value representative of the Percent Bias is less than 0.1 mv.

TABLE 7
Comparison of Newly-made Solid-State Reference Electrode

| | Reference | Newly-made | Percent Bias | |
|---|---|---|---|---|
| Samples | values | sensors | (absolute | Millivolt |
| # | (mM) | (mM) | value) | Difference |
| 1 | 144 | 142 | 1.39% | 0.056 |
| 2 | 137 | 137 | 0.00% | 0.000 |
| 3 | 140 | 141 | 0.71% | 0.029 |
| 4 | 134 | 133 | 0.75% | 0.030 |
| 5 | 149 | 150 | 0.67% | 0.027 |
| 6 | 147 | 145 | 1.36% | 0.054 |
| 7 | 146 | 143 | 2.05% | 0.082 |
| 8 | 132 | 131 | 0.76% | 0.030 |
| 9 | 152 | 149 | 1.97% | 0.079 |
| 10 | 141 | 140 | 0.71% | 0.028 |

| | | |
|---|---|---|
| Average Value | 1.04% | 0.042 |
| Maximum Value | 2.05% | 0.082 |

TABLE 8
Comparison of Concentration Reference Value
when Solid-State Reference Electrode is
Stored for 6 Months at Room Temperature

| | | Sensors - | | |
|---|---|---|---|---|
| | | Half year | | |
| | | storage at | | |
| | Reference | room | Percent Bias | |
| Samples | values | temperature | (absolute | Millivolt |
| # | (mM) | (mM) | value) | Difference |
| 1 | 144 | 144 | 0.00% | 0.000 |
| 2 | 137 | 136 | 0.73% | 0.029 |
| 3 | 140 | 138 | 1.43% | 0.057 |
| 4 | 134 | 135 | 0.75% | 0.030 |
| 5 | 149 | 147 | 1.34% | 0.054 |
| 6 | 147 | 146 | 0.68% | 0.027 |
| 7 | 146 | 144 | 1.37% | 0.055 |
| 8 | 132 | 134 | 1.52% | 0.061 |
| 9 | 152 | 151 | 0.66% | 0.026 |
| 10 | 141 | 143 | 1.42% | 0.057 |

| | | |
|---|---|---|
| Average Value | 0.99% | 0.040 |
| Maximum Value | 1.52% | 0.061 |

TABLE 9
Comparison of Concentration Reference Value
when Solid-State Reference Electrode is Stored
for 6 Months at 4° C. Temperature

| | | Sensors - | | |
|---|---|---|---|---|
| | | Half year | | |
| | | storage | | |
| | Reference | at 4° C. | Percent Bias | |
| Samples | values | temperature | (absolute | Millivolt |
| # | (mM) | (mM) | value) | Difference |
| 1 | 144 | 142 | 1.39% | 0.056 |
| 2 | 137 | 138 | 0.73% | 0.029 |
| 3 | 140 | 138 | 1.43% | 0.057 |
| 4 | 134 | 134 | 0.00% | 0.000 |
| 5 | 149 | 148 | 0.67% | 0.027 |
| 6 | 147 | 146 | 0.68% | 0.027 |
| 7 | 146 | 145 | 0.68% | 0.027 |
| 8 | 132 | 133 | 0.76% | 0.030 |
| 9 | 152 | 152 | 0.00% | 0.000 |
| 10 | 141 | 141 | 0.00% | 0.000 |

| | | |
|---|---|---|
| Average Value | 0.63% | 0.025 |
| Maximum Value | 1.43% | 0.057 |

TABLE 10
Comparison of Concentration Reference Value when Solid-State
Reference Electrode is Stored for 1 year at Room Temperature

| | | Sensors - | | |
|---|---|---|---|---|
| | | one year | | |
| | | storage at | | |
| | Reference | room | Percent Bias | |
| Samples | values | temperature | (absolute | Millivolt |
| # | (mM) | (mM) | value) | Difference |
| 1 | 144 | 141 | 2.08% | 0.083 |
| 2 | 137 | 136 | 0.73% | 0.029 |
| 3 | 140 | 142 | 1.43% | 0.057 |
| 4 | 134 | 132 | 1.49% | 0.060 |
| 5 | 149 | 151 | 1.34% | 0.054 |
| 6 | 147 | 148 | 0.68% | 0.027 |
| 7 | 146 | 145 | 0.68% | 0.027 |
| 8 | 132 | 131 | 0.76% | 0.030 |
| 9 | 152 | 153 | 0.66% | 0.026 |
| 10 | 141 | 142 | 0.71% | 0.028 |

| | | |
|---|---|---|
| Average Value | 1.06% | 0.042 |
| Maximum Value | 2.08% | 0.083 |

TABLE 11
Comparison of Concentration Reference Value when Solid-State
Reference Electrode is Stored for 1 year at 4° C. Temperature

| | | Sensor - | | |
|---|---|---|---|---|
| | | one year | | |
| | | storage | | |
| | Reference | at 4° C. | Percent Bias | |
| Samples | values | temperature | (absolute | Millivolt |
| # | (mM) | (mM) | value) | Difference |
| 1 | 144 | 145 | 0.69% | 0.028 |
| 2 | 137 | 136 | 0.73% | 0.029 |
| 3 | 140 | 140 | 0.00% | 0.000 |
| 4 | 134 | 133 | 0.75% | 0.030 |
| 5 | 149 | 150 | 0.67% | 0.027 |
| 6 | 147 | 146 | 0.68% | 0.027 |
| 7 | 146 | 144 | 1.37% | 0.055 |
| 8 | 132 | 133 | 0.76% | 0.030 |
| 9 | 152 | 154 | 1.32% | 0.053 |
| 10 | 141 | 142 | 0.71% | 0.028 |

| | | |
|---|---|---|
| Average Value | 0.77% | 0.031 |
| Maximum Value | 1.37% | 0.055 |

Although the preferred embodiments of the present invention have been described herein, the above description is merely illustrative. Further modification of the invention herein disclosed will occur to those skilled in the respective arts and all such modifications are deemed to be within the scope of the invention as defined by the appended claims.

1. A solid-state reference electrode for use in whole blood, serum, plasma, other biological fluids, and/or aqueous solutions, the solid-state reference electrode comprising:
an insulating support substrate;
an electrically-conductive material disposed on the insulating support substrate, wherein a first portion of the electrically-conductive material is an electrode portion and a second portion of the electrically-conductive material is an electrical contact portion, the electrically-conducting material comprising one of a conductive noble metal, an electrically conductive ink, or a metal-metal salt selected from the group consisting of silver-silver chloride and mercury-mercurous chloride;
an electrode-forming insulating layer having an opening, the electrode-forming insulating layer disposed on the insulating substrate layer, wherein the opening forms a well having predefined dimensions and exposing the electrode portion of the electrically-conductive material;
a metal-metal salt layer disposed in the well over the electrode portion of the electrically-conductive material when the electrically-conductive material is not the metal-metal salt; and
a hydrogel polymeric membrane disposed (a) on the metal-metal salt layer forming the solid-state reference electrode when the electrically-conductive material is not the metal-metal salt, or (b) directly on the electrically-conductive material when the electrically-conductive material is the metal-metal salt, the hydrogel polymeric membrane being a polymeric hydrogel network containing (i) a chloride salt from inorganic salts of chlorides or organic salts of chlorides and (ii) a supporting electrolyte from salts of an anionic species.

2. The solid-state reference electrode as claimed in claim 1 further comprising a second polymeric membrane disposed onto the hydrogel polymeric membrane, the second polymeric membrane selected from hydrophilic silicon compounds or from lipophilic polymers.

3. The solid-state reference electrode as claimed in claim 1, wherein the conductive noble metal is selected from the group consisting of gold, platinum, palladium, copper, indium, and tin oxide.

4. The solid-state reference electrode as claimed in claim 1, wherein the chloride salt in the hydrogel polymeric membrane is selected from the group consisting of sodium chloride, potassium chloride, lithium chloride, choline chloride, 1-butyl-3-methylimidazolium chloride, 1-butyl-2,3-dimethylimidazolium chloride, 1-butyl-1-methylpyrrolidium chloride, 1,2-dimethyl-3-propylimidazolium chloride, and 1,3-dimethylimidazolium chloride.

5. The solid-state reference electrode as claimed in claim 1, wherein the supporting electrolyte is selected from the group consisting of lithium, sodium or potassium salts of citrates, acetates, sulfonates, or triflates.

6. The solid-state reference electrode as claimed in claim 2, wherein the silicone compounds are selected from the group consisting of diluted silicon tetrachloride, aminopropyltriethoxysilane, n-[3-(trimethoxysilyl)propyl]ethylenediamine, methyltrimethoxy silane, and phenyltrimethoxysilane.

7. The solid-state reference electrode as claimed in claim 2, wherein the lipophilic polymers are selected from the group consisting of polyvinylpyrrolidone, polyvinyl alcohol, PVC, poly(methyl methacrylate), agar, gelatin, poly(urethane), cellulose acetate butyrate, cellulose acetate, and nitro cellulose.

8. The solid-state reference electrode as claimed in claim 2, wherein the metal-metal salt layer is silver-silver chloride, the chloride salt in the hydrogel polymeric membrane is potassium chloride, and the supporting electrolyte is lithium acetate.

9. The solid-state reference electrode as claimed in claim 1, wherein the hydrogel polymeric membrane is selected from the group consisting of polyacrylates, polymethacrylates, polyvinyl compounds, polyurethanes, polycarbamoyl sulfonates, polyureas, polyethers, crosslinkable PVA-SBQ, crosslinked protein matrix like gelatin, silk fibroin, BSA, crosslinked polysacharrides like cellulose, dextrans, cyclodextrans, alginates, chitosan, Agar, and any combination of thereof.

10. The solid-state reference electrode as claimed in claim 1, wherein the hydrogel polymeric membrane further includes (a) a hydrophilic plasticizer capable of filing the polymeric hydrogel network and solidifying and plasticizing the hydrogel polymeric membrane, and (b) a high molecular weight polymer capable of reinforcing the polymeric hydrogel network.

11. The solid-state reference electrode as claimed in claim 10, wherein the hydrophilic plasticizer is selected from at least one of glycerol, polyethylene glycol, ethylene glycol monomethyl ester, ethylene glycol, and formamide.

12. The solid-state reference electrode as claimed in claim 10, wherein the high molecular weight polymer is one of polyethylene glycol, polyvinyl alcohol, polyvinylpyrrolidone, poly(2-hydroxyethyl methacrylate), and particulates like pyrogenic silica or latex.

13. The solid-state reference electrode as claimed in claim 1, wherein the hydrogel polymeric membrane includes poly-2-hydroxyethyl methacrylate, ethylene glycol as the hydrophilic plasticizer, and polyvinylpyrrolidone as the high molecular weight polymer.

14. A method of making a solid-state reference electrode for use in whole blood, serum, plasma, other biological fluids, and/or aqueous solutions, the method comprising:
obtaining either (a) an insulating support substrate having an electrically-conductive material disposed on at least one side of the insulating support substrate, or (b) an insulating support substrate and disposing an electrically-conductive material onto at least one side of the insulating support substrate wherein a first portion of the electrically-conductive material is an electrode portion and a second portion of the electrically-conductive material is an electrical contact portion, the electrically-conducting material in (a) and (b) comprising one of a conductive noble metal, an electrically conductive ink, or a metal-metal salt selected from the group consisting of silver-silver chloride and mercury-mercurous chloride;
disposing an electrode-forming insulating layer having an opening onto the insulating support substrate, the opening forming a well, wherein the well exposes the electrode portion of the electrically-conductive material;
forming a metal-metal salt layer on the electrode portion of the electrically-conductive material exposed in the well when the electrically-conductive material is not the metal-metal salt;
disposing a predefined amount of a precursor solution into the well, the precursor solution containing a chloride salt solution, a supporting electrolyte solution, a hydrogel polymer, a hydrophilic plasticizer, a high molecular weight polymer, a cross-linking reagent, and a radical initiator; and
exposing the precursor solution in the well to radiation forming a hydrogel polymeric membrane and thereby forming the solid-state reference electrode.

15. The method of claim 14, wherein the forming step includes forming a silver-silver chloride layer.

16. The method of claim 14 further includes comprising:
forming the precursor solution by mixing a predefined amount of a chloride salt solution, a predefined amount of a supporting electrolyte solution, a predefined amount of hydroxyethyl methacrylate, a predefined amount of ethylene glycol, a predefined amount of polyvinylpyrrolidone, a predefined amount of tetraethylene glycol dimethacrylate, and a predefined amount of 2,2-dimethoxy-2-phenylacetophenone.

17. The method of claim 14 further comprising:
forming the precursor solution by mixing an amount in a range selected from the group consisting of 20-80 wt. %, 30-70 wt. %, and 45-55 wt. % of poly-2-hydroxyethyl methacrylate as the cross-linkable hydrogel polymer, an amount in a range selected from the group consisting of 20-80 wt. %, 30-60 wt. %, and 40-50 wt. % of ethylene glycol as the hydrophilic plasticizer, an amount in a range selected from the group consisting of 0.5-10.0 wt. % and 1-5 wt. % of polyvinylpyrrolidone as the high molecular weight polymer, an amount in a range selected from the group consisting of 0.1-2.0 wt. % and 0.5-1.0 wt. % of tetraethylene glycol dimethacrylate as the cross-linking reagent, and an amount in the range selected from the group consisting of 0.01-2.0 wt. % and 0.5-1.0 wt. % of 2,2-dimethoxy-2-phenylacetophenone as the radical initiator.

18. A method of making the hydrogel polymeric membrane of claim 1 for use in a solid-state reference electrode, the method comprising:
forming a precursor solution comprising:
a predefined amount of a chloride salt;
a predefined amount of a supporting electrolyte salt;
a predefined amount of cross-linkable hydrogel polymer;
a predefined amount of hydrophilic plasticizer;
a predefined amount of high molecular weight polymer;
a predefined amount of a cross-linking reagent; and
a predefined amount of a radical initiator;
disposing a predefined amount of the precursor solution onto a solid-state electrode containing a metal-metal salt electrolytic electrode; and
photo-irradiating the predefined amount of the precursor solution to ultraviolet light.

19. The method of claim 18 further comprising:
selecting potassium chloride as the chloride salt;
selecting lithium acetate as the supporting electrolyte salt;
selecting poly-2-hydroxyethyl methacrylate as the cross-linkable hydrogel polymer;
selecting ethylene glycol as the hydrophilic plasticizer;
selecting polyvinylpyrrolidone as the high molecular weight polymer;
selecting tetraethylene glycol dimethacrylate as the cross-linking reagent; and
selecting 2,2-dimethoxy-2-phenylacetophenone as the radical initiator.

20. The method of claim 18 further comprising:
selecting an amount in a range from the group consisting of 0.1 mM to saturated, 10 mM to 500 mM, and a concentration of 200 mM of the chloride salt;
selecting an amount in a range from the group consisting of 10 mM to saturated, 1M to 6 M, and 3 M to 5M of the supporting electrolyte salt;
selecting an amount in a range from the group consisting of 20-80 wt. %, 30-70 wt. %, and 45-55 wt. % of poly-2-hydroxyethyl methacrylate as the cross-linkable hydrogel polymer;
selecting an amount in a range from the group consisting of 20-80 wt. %, 30-60 wt. %, and 40-50 wt. % of ethylene glycol as the hydrophilic plasticizer;
selecting an amount in a range from the group consisting of 0.5-10.0 wt. % and 1-5 wt. % of polyvinylpyrrolidone as the high molecular weight polymer;
selecting an amount in a range from the group consisting of 0.1-2.0 wt. % and 0.5-1.0 wt. % of tetraethylene glycol dimethacrylate as the cross-linking reagent; and
selecting an amount in the range from the group consisting of 0.01-2.0 wt. % and 0.5-1.0 wt. % of 2,2-dimethoxy-2-phenylacetophenone as the radical initiator.
